(19) United States
(12) Patent Application Publication
(10) Pub. No.: US 2004/0049129 A1
(43) Pub. Date: Mar. 11, 2004

(54) Wafer assembly having a resilient extendable/retractable needle means for healthcare

(76) Inventor: Yan Qi, Highlands Ranch, CO (US)

Correspondence Address: Yan Qi, 421 E. Bexley Lane, Highlands Ranch, CO, 80126, US

(21) Appl. No.: 10/235,384

(22) Filed: Sep. 5, 2002

(51) Int. Cl. A61B 5/00

(52) U.S. Cl. 600/576

(57) ABSTRACT

A wafer assembly having a resilient extendable/retractable needle addresses the problems of safety and convenience in needle techniques. The “L” shaped resilient needle, which disposed within a housing pod of the assembly, is able to resiliently extend from the housing pod to puncture a subject and able to resiliently retract within the housing pod after puncturing. The wafer assembly contacts the subject that including adheres to the subject. The invention assembly avoids any potential risks associated with needle techniques that include needle-stick, needle irritation, contamination and cross-infection. The assembly enables a preset application, which includes a test application or a delivery application, in such assembly. It may be available used one continuous motion to punch the needle, to perform the preset application, and conceal the needle. The assembly is of simple mechanism to easily make with economic benefits and to conveniently perform with fewer malfunctions.

### BACKGROUND OF THE INVENTION

Due to increasing health risks in the areas of healthcare (e.g. medicine and biotechnology), a higher degree of awareness has arisen concerning problems associated with needle techniques (e.g. withdrawal and injection delivery techniques). For example, occupational risks in the clinical setting for infectious agents such as hepatitis B and C, HIV, and other pathogens have generated a demand for safer techniques. Usually, current methods for withdrawal of sample or delivery of pharmacological agents require a high degree of professional training, (e.g. by a doctor or nurse) and remain high risk of needle-stick. However, with homecare becoming more prevalent in developed countries, the demands for safer and more convenient techniques will be even more important in the future. Patented technologies have been developed to address the problems inherent in current withdrawal/delivery techniques. However, the designs of devices developed to date do not fully address safety and convenience issues.

In prior patents, the device includes an L-shaped needle (U.S. Pat. Nos. 4,235,234, 5,993,423, 5,858,001, and 6,074,369) for delivery, the needle being integrally joined with a needle holder or a needle carrier. The purpose of this design is to fix the needle, and thus preventing a breakage of the needle as the holder is pressed against the skin of the subject. The L-shaped injection needle stays in the patent's tissue for delivery of the drug. However, because the L-shaped injection needle has an exposed needle for injection, it also carries risks associated with accidental needle-sticks. Further, the L-shaped injection needle itself does not have an automated retracting ability to reduce continued trauma to the injection site after delivery. Also, these devices do not help to stop bleeding or protect the injection site.

Safer devices that involve retractable syringes or syringes-like devices have been developed in order to address the issue of accidental needle-sticks. These devices usually house the needle, which is held by a needle holder, before and/or after the actual insertion process. In this case, the sharp end of the needle is only exposed during actual withdrawal or delivery. However, these devices still use a traditional apparatus with an exposed needle, and there remains a certain level of risk for accidental needle-sticks.

The previously devices may have shortcomings regarding safety. That is, these devices involve an exposed needle during a medical procedure. The medical procedure includes before the process of insertion, or during the process of insertion, or after the process of insertion. The devices are problematic for two reasons: 1. The exposed needle may cause accidental needle-sticks. 2. The blood or body fluid from the inserted site of the subject may become a source of contamination and cross-infection. Furthermore, these current devices do not protect the inserted site immediately after insertion to avoid risks associated with fluids from the subject (e.g. blood or body fluid).

In addition to the safety issues described above, there is also a need for convenience in a device for healthcare (e.g. withdrawal or delivery). The device is not only available used by trained professed person but also used by non-professed person.

### SUMMARY OF THE INVENTION

In view of the foregoing, an object of the present invention is to provide a healthcare device, which includes a test or delivery device that addresses the problems of safety and convenience in needle techniques. The object of present invention is of simple mechanism that easily makes with economic benefits and easily performs with fewer malfunctions.

This and other objects of the present invention are achieved by providing a device having a resilient extendable/retractable needle means, and a dressing portion that is in contact with the subject's surface.

In one embodiment, the present invention provides a wafer assembly having the resilient extendable/retractable punching mechanism, at least one application, and a dressing portion. The wafer assembly dresses the target subject surface before insertion, inserts the needle into the subject to perform the application task, automatically retracts the needle back to a safe position after insertion, and protects the wound at the inserted site. The application includes an application container; here the wafer assembly inserts the needle into the subject to perform the task (e.g., withdrawal of a testing sample, or delivery of a drug) specified by the application container.

In one suitable approach, the present invention may use a resilient needle, which may be an “L” shaped sterilized needle, and which may be housed within the assembly. The needle has extending/retracting ability relative to the assembly. The extending/retracting ability of the needle allowing the sharp end of the needle to be exposed to the subject only during insertion, and allows the sharp end of the needle to be protected within the assembly before and after insertion. Further, because the needle is automatically retracted back into the assembly after insertion, the assembly prevents continued trauma and contamination in relation with the needle.

In one suitable approach, the present invention may dress the surface of the target subject to provide protection to the insertion site. The wafer assembly may, like a bandage, be a thin and lightweight article that conveniently dresses the insertion site.

In one suitable approach, the present invention may provide at least one application container in the assembly for test or delivery task, the application container being filled with either a substance for delivery, or a set test component for test sample. The application container may use one continuous motion to insert the needle, to perform the preset application, and to conceal the needle after insertion. In this case, the assembly may be able to perform its function automatically and without further human action after being triggered.

In one suitable approach, the present invention may provide a disposable assembly.

In one suitable approach, the present invention may provide an assembly that signifies its status (e.g., before, during or after use of the assembly).

In one embodiment, the present invention may combine the assembly with an associated device having an operating mechanism and a measuring mechanism. Said device may be either a portable or a fixed device. The assembly may be interchangeably attached to the device. The device may be a tool for easily using the assembly. The device may only have an operating mechanism that may be coupled to the assembly, which may activate the assembly. Or the device may have only a measuring mechanism to do detection on the assembly.

In one suitable approach, the present invention may enable input and output of applicable information to and from the associated device via an Internet interface.

In one embodiment, the present invention may provide a disposable assembly to a completely automated system for performing the task of assembly e.g. test or delivery.

### BRIEF DESCRIPTION OF THE DRAWINGS

FIG. 1A is a sectional view of one assembled embodiment with a resilient extendable/retractable needle set in.

FIG. 1B is a sectional view of one assembled embodiment with an associated device.

FIG. 2 is an exploded perspective view of one embodiment with an action groove and an action bar.

FIG. 3 is sectional view of assemblies with different shape of the needles and others. 3A. 3B. 3C .3D

FIG. 4A is a sectional view of one embodiment with actuation means that a trigger button with a locker.

FIG. 4B is a sectional view of one embodiment with actuation means that a trigger button without a locker.

FIG. 4C is a sectional view of one embodiment with actuation means that a trigger button with two push-membaers.

FIG. 5A is an exploded perspective view of one embodiment with a test container.

FIG. 5B is an exploded perspective view of one embodiment with a test container and a mini-pump.

FIG. 6A is an exploded perspective view of one embodiment with a U shaped integrate action bar.

FIG. 6B is an exploded perspective view of one embodiment with a bar shaped integrate action bar.

FIG. 7 is an exploded perspective view of one embodiment, one integrate action bar with a pump services as application container.

FIG. 8A is an exploded perspective view of one embodiment, one closed integrate action bar services as application container for delivery task.

FIG. 8B is an exploded perspective view of one embodiment, one closed integrate action bar services as application container for test task.

### DETAILED DESCRIPTION OF THE INVENTION

In order to better understand the invention, the following detailed description of its parts and functions are given. The following preferred embodiments are described with drawings, with the understanding that these drawings depict only construct embodiments of the invention and are not limited to the scope.

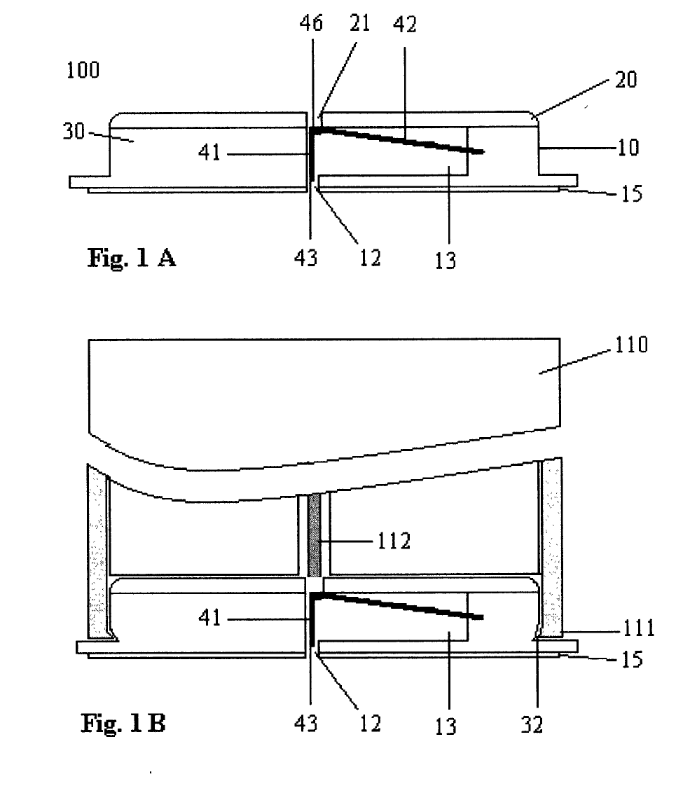

In one embodiment, the present invention may provide a wafer assembly to be dressed on the surface of the subject. As shown in FIGS. 1-3, assembly 100 is a basic wafer assembly having a resilient extendable/retractable needle mechanism. Pod 30 may comprise a base member 10 and a top member 20 integrated into one piece. The base member 10 has two major surfaces, low-surface 11 and up-surface 14. Up-surface 14 of the pod base member has a small needle-groove 13 for setting the resilient extendable/retractable needle 40. The needle-groove 13 preferably has a rectangular cross-section, but is not limited to a rectangular cross-section. The size of the needle may match the size of the groove that guides the needle's up-down motion. The base member maybe has a dress portion 15 at its low-surface 11 that may adhere to the subject's surface. On low-surface 11 of the base member there is a hole 12 in the needle-groove that acts as a pathway for the needle to insert into the subject 1. The top member 20 has a small open 21 which may be used to initiate the function of the assembly. For example, the small open 21 may house either a trigger button 90 or a perform-member that may be from an associated device.

The extendable/retractable needle may be a resilient needle 40, which may comprise short-arm 41 and long-arm 42, as an “L” shaped needle. The long-arm 42 may be a springy arm. Usually the needle is an angle bend needle which the bend tip as the short-arm and the major body as the long-arm. The short-arm of needle has a vertical location relative with-the pod 30. One end of the short arm 41 of the needle is sharp end 43, for insertion into the subject 1 via a pushing force from the opposite end of the needle, called push-end 46. Preferably the push-end 46 is located on the junction point of the short arm 41 and the long-arm 42. The short arm of the needle moves toward the direction of the subject to extend out the pod 30 when the push-end 46 is pushed. Because the long-arm 42 is in a major axis of said needle groove as the spring arm is fixed on pod at its end, the needle 40 is stand into the needle-groove 13 of pod. When the short-arm of the needle moves up or down, it is dependent on changes in position and/or shape of the springy long-arm. Prior to punching, the needle 40 is kept at a position called initial position. The initial position means that the needle 40 touches the top member of the pod via spring support from the long-arm 42. Because the short arm 41 is a bit shorter than the thickness of the assembly, the needle is concealed into the needle-groove 13 of the pod without the sharp end 43 being exposed. The extending/retracting ability of needle 40 means that the needle allows insertion into the subject 1 via push power (horizontal push power or vertical push power) and allows automatic retraction back into the pod via spring power of the long-arm after the push power is withdrawn.

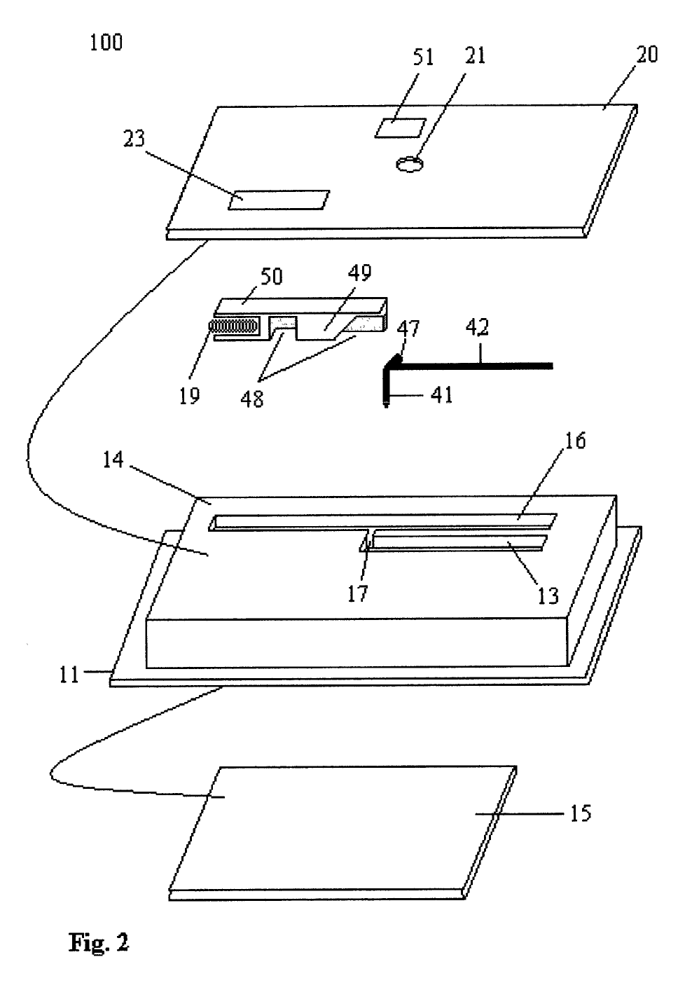

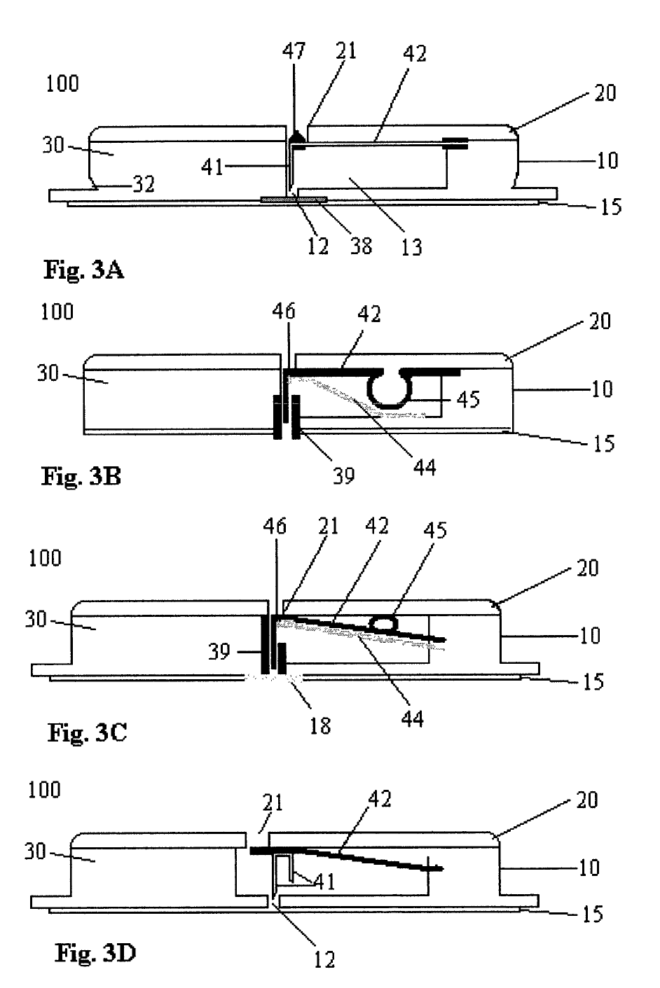

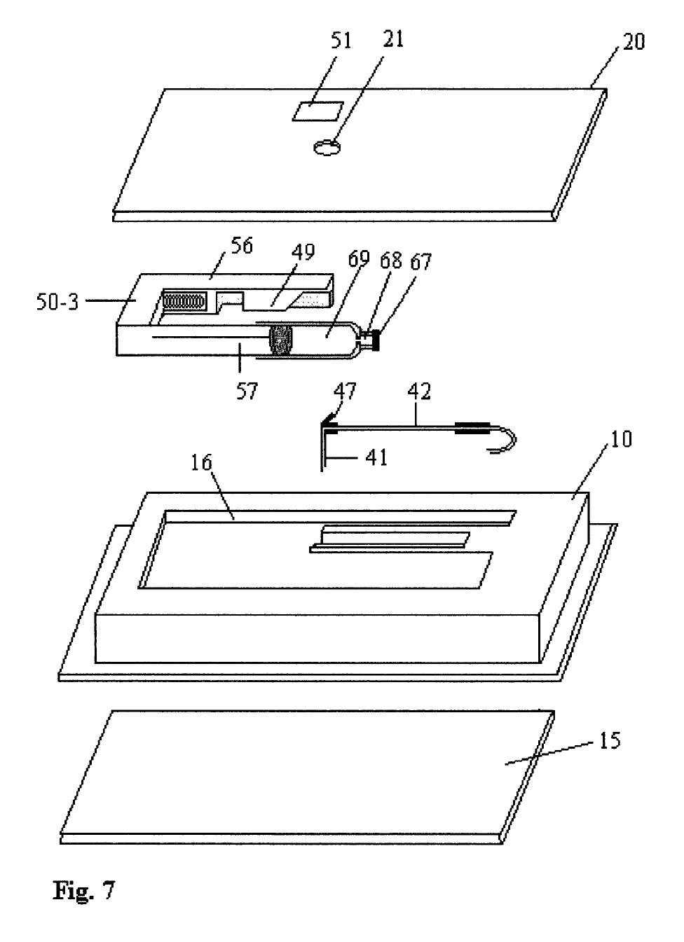

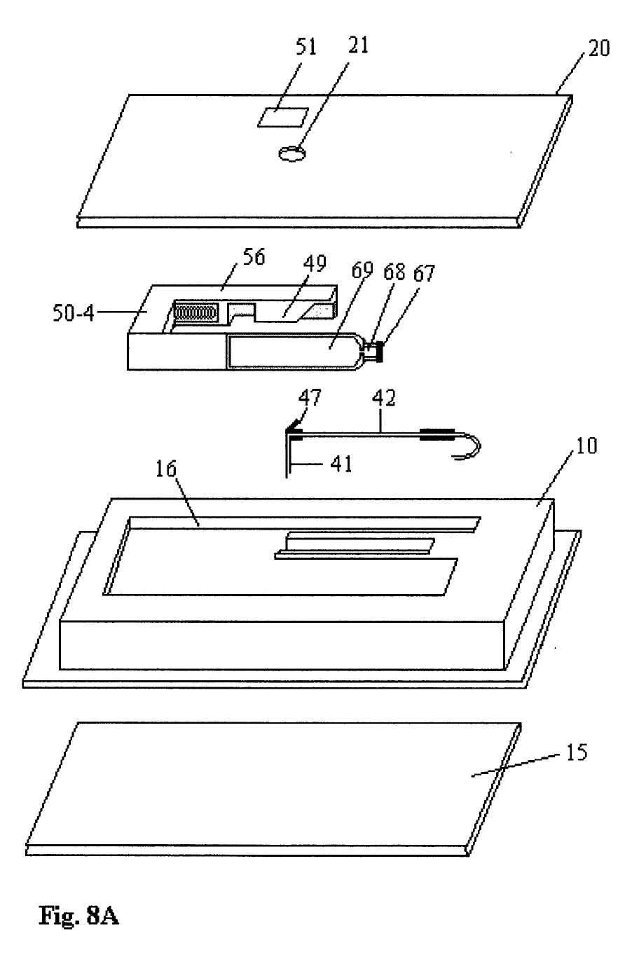

In most cases, the push end of needle 46 may have a protrusion 47 that is pushed. In some cases, the needle may not have the protrusion. The protrusion 47 could be a branch of the long-arm (as shown in FIG. 2) or the protrusion 47 may be an independent protruding part to be fixed to the needle (as shown in FIG. 3A, FIG. 7). The protrusion 47 may have two positions on the long-arm, either at the side of the long-arm or on top of the long-arm. The needle 40 may move followed the movement of the protrusion. When the protrusion 47 forms at the side of the long-arm, there may be a slot or space 17 in the base member of the pod for the protrusion's up-down motion. The slot or space 17 may be independent if the protrusion 47 is configured like a tree branch on the long-arm. The slot or space 17 may be a part of the needle groove if the protrusion 47 is one the same axis as the long-arm (as shown in FIG. 3D). When the protrusion 47 forms at the top of long-arm, the protruding goes with needle 40 into the needle groove. Further, the spring long-arm 42 maybe have a circular spring element 45 to make the short arm more flexible in its up-down movement. Said circular spring element also changes the length of the long-arm in the insertion process, and lets the short-arm insert into the subject with preferably degree. The spring element 45 may be an unclosed U shape (shown in FIG. 3B) or a spiral shape (shown in FIG. 3C). And further, the spring power which keeps the needle at its initial position and which conceals the needle into the pod after insertion is not supported by said spring long-arm 42 alone. An associate spring-piece 44 may be included to support the spring long-arm. Said spring-piece 44, one end of which may be fixed to the needle, may be set into needle-groove 13 as well (shown in FIG. 3B or 3C).

The base member may include a ring member 39 located at the hole 12 in the needle-groove 13 (shown in FIG. 3C). The ring member 39 may be disposed around the short-arm of needle 41, and may define the path of the needle. Because the short-arm of needle 41 moves in the ring's interior, the ring member 39 may be constructed from firm material that allows guidance of the direction of movement for the short arm. It is especially useful for a non-vertical punch (less than 90 degree) on the subject and for insertion of a fine needle into the subject. Said ring member 39 maybe over the low-surface of base member 11 (shown in FIG. 3B). When the ring member reaches to the top member 20, the ring member has an opening at the side of the ring member to fit the needle's long-arm up-down motion.

In some kinds of assemblies, there may be a penetrable member 38 that sealed the hole 12 of the base member (shown on FIG. 3A). The penetrable member, (e.g., thin rubber piece) may prevent blood, body fluid of the subject or treating regent into the assembly. For fitting the penetrable member 38 there is a shallow space on the low surface of the base member.

In one suitable approach, the assembly may provide a dress portion 15 on the low-surface 11 of base member 10. Said dress portion means may attach to the subject surface by direct adhesion or by vacuum suction (via a preset vacuum power). The assembly dresses on a subject 1 allowing for protection of the punch site. The protection of the punch site has benefits for avoiding contamination at the punch site. In the non-live subject, the assembly usually is sealed or stuck to the subject surface permanently. Said here, the subject surface for the assembly includes the packaging material of subject, which means that the assembly may be fused or stuck to the package of the subject, including a membrane package and a penetrable container for the subject (e.g., drug, special biological drug, milk and some foods). The assembly may be used on the package surface to perform testing or delivery. In the live subject, the assembly is usually stuck on subject surface temporarily to protect the wound of punch site. This means that the assembly can be taken from the live subject after protection is no longer needed like a conventional bandage. About the dress portion 15, the portion at the base member may include materials for adhesion to the subject. The dress portion may also include anesthetic and antibiotic reagent. Said anesthetic reagent for reducing the pain in the punching and said anti-biotic reagent for reducing the risk of infection at the punch site.

The area around the hole 12 is called the center area, whether the center area has an adhesive or non-adhesive may depend on the application container present inside the pod. In some cases, the center area has a thin pad 18 laid on the low surface 11 to protect the punch site and take some leaking sample e.g. blood sample. Said thin pad 18 is an absorbent material pad. Usually the thin pad 18 is a very small size and located at the hole 12 that does not have adhesive material on it (shown in FIG. 3C). Further, the base member may also have a winger type with adhesive to stick on the subject. For easily taking off the assembly from subject, the winger type may have some area around its edges without adhesive material. Further, the dress portion 15 is available to be delivered between the lower surface of the base member and the subject by a preset mini-pump that is filled with adhesive material.

The needle is pushed by either a horizontal moving power or a vertical moving power. In the horizontal case, there is an action-groove 16 in the pod. The action-groove 16 houses a horizontal action-bar 50. The action-groove 16 preferably has a rectangular cross-section. The shape and size of the action-bar 50 matches to the action-groove 16 and lets the action-bar moves in the action-groove smoothly. Said the action-groove 16 is located in a pod that includes the base member 10 and/or the top member 20. The action groove 16 communicates with the slot 17 to fit the protrusion 47 of needle coming into the action-groove 16. The horizontal action-bar 50 moves into the action-groove to push the protrusion 47 toward the subject. When said action-groove crosses to said needle groove in the pod, said long-arm 42 works as said protrusion to be pushed down (not shown). In general, the action-bar 50 pushes the protrusion 47 to turn the horizontal movement of the action-bar into the vertical movement of the short-arm of the needle.

The horizontal action-bar 50 has a push-part 49 and at least one cavity 48. The needle 40 is inserted via the push-part 49 and concealed back the pod via the cavity 48. Said push-part 49 is a protrusion, which is within cavity or with cavity 48, to be used to push the needle. As described before, if the needle has the protrusion 47, it is either at the side or on top of the long-arm of the needle.

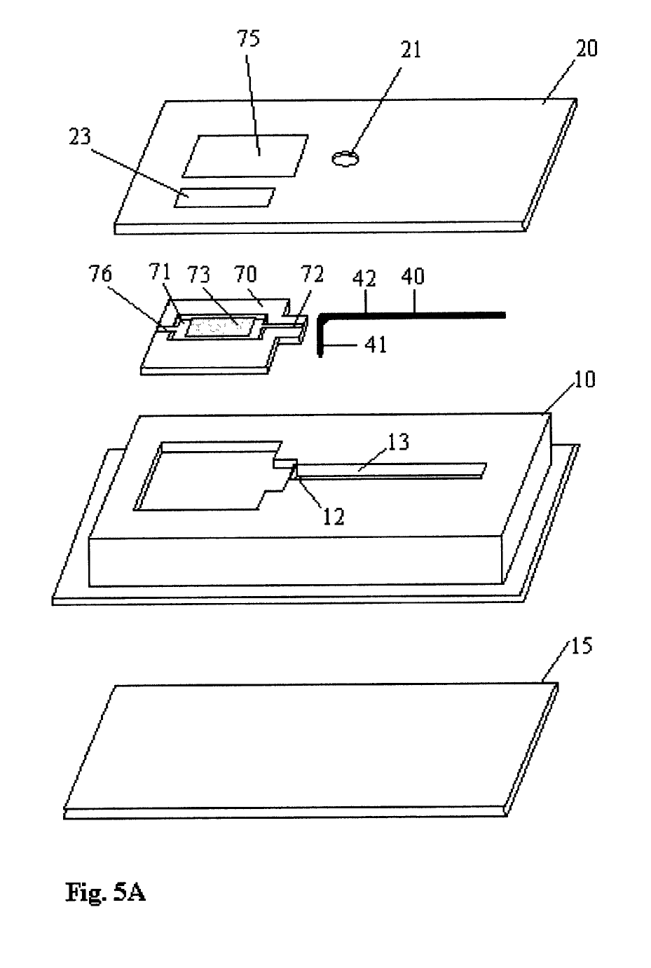

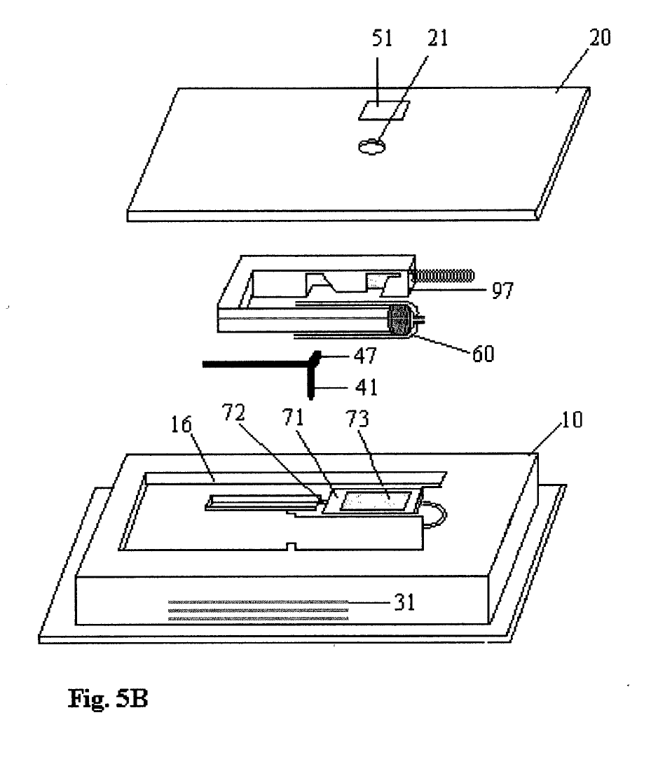

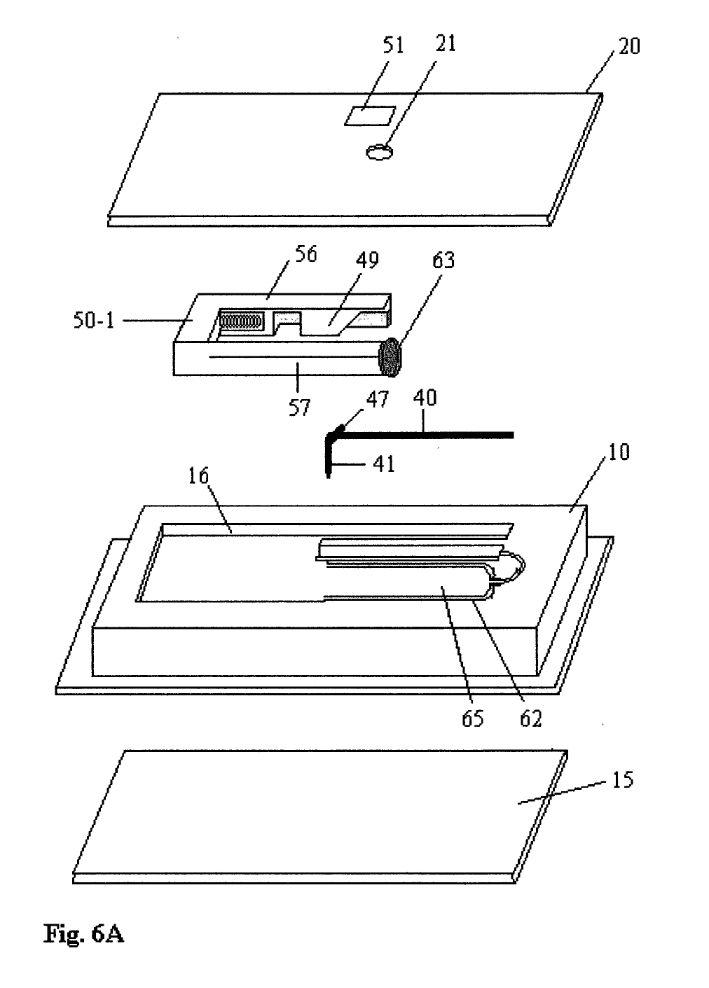

When the protrusion 47 forms on the side of the long-arm, the horizontal action-groove 16 may be located in the base member 10. Usually, the needle groove 13 and the action-groove 16 are parallel-grooves; the slot 17 communicates with the grooves for protrusion 47 into the action-groove 16. The movement of the short-arm of the needle follows the movement of the protrusion 47. Here, the push-part 49 and cavity (or cavities) 48 of the action-bar are located at the side of the action-bar for the up-down motion of the side protrusion. Further, in some case, the movement of action-bar is available used to lock the needle in the pod after the punch done. Here the rear edge of the cavity is a sliding surface 97 faces to the protrusion 47. Followed the action-bar, the sliding surface at the rear edge of the cavity could push the protrusion 47 back to needle groove and lock the protrusion 47 into the pod of assembly (shown in FIG. 5B).

When the protrusion 47 forms on the top of the long-arm, the action bar moves into the action groove 16 of the pod (which may be in the top member 20). The needle groove 13 and the action groove 16 communicate at the lower surface of the action groove 16 where protrusion 47 runs into the action groove 16 (not shown). In here, the push-part 49 and cavity (or cavities) 48 of action-bar are located at the lower surface of the action bar.

For the action-bar to move easily to push the needle down, the protrusion 47 and the push-part 49 have a smooth contact. This means the shape of the push-part matches with the shape of protrusion to ensure easy sliding. For example 1: the protrusion 47 is a thin cylinder and the contact surface of the push-part 49 is a sliding surface that matches to the cylinder. For example 2: the protrusion 47 is a cone, and the contact surface of the push-part 49 is a cone shaped sliding surface that matches the cone. However the contact surface of the push-part is a smooth sliding surface that faces the subject and enables pressing of the needle into the subject. The cavity 48 may be cut-out space, where the needle 40 may be concealed with the pod to avoid exposing the sharp end 43. The cavity 48 may be either a small cut-out space or a long cut-out space. The small cut-out space of cavity 48 allows the protrusion 47 back and stops the horizontal action-bar from moving. The long cut-out space allows protrusion 47 back into the space to let the horizontal action bar to continue moving. The size of the push-part depends on the purpose of assembly. For example, in an assembly having a preset application container for test or delivery, the action-bar moving within action groove 46 is not only to push the needle into the subject and back into the pod but also maybe to perform the preset application. Basic on the requirement of the application, the size of push part 49 on the action bar would be different. In the case of a small volume blood testing (e.g. blood glucose test), the needle only briefly punctures the skin of the patient before retracting back. So the push-part 49 is a relatively smaller size. The push-part 49 may have a triangle cross-section. In an injection case, the needle needs to stay in the subject until the injection process is done, so the push part is a relatively larger size. The depth the needle inserts into the subject depends on the thickness of the horizontal action-bar's push-part 49. Further, when the assembly has the spring piece 44 to support the needle, the inventive assembly may also include a protrusion on the spring piece 44 to provide a function like that of protrusion 47.

Further, about assembly there is an action-window 51 at the top member. Said action-window shows action-bar's position that relates with movement of the action bar. If the action bar has an indicator on its body, the relation of the window 51 and the action-bar 50 may be used easily to show the assembly's status of use, i.e. before, during and after use. Preferably the indicator is a color on the action bar at different positions, for example: green shows before use, yellow shows during use and red shows after use. Because the action bar moves only in one direction, the sharp end of the needle is never exposed after the assembly is used.

The movement of action-bar is made by a preset movement mechanism or by a push power from outside of the assembly.

In one suitable approach, the assembly has a movement mechanism built into the pod 30. The movement of the action-bar is performed by the movement mechanism after being triggered. The movement mechanism comprises a power means and a trigger button 90. The action-bar is moved by one of the power means including a spring power, rubber band power, an electric power, an air power, and a hydraulic power.

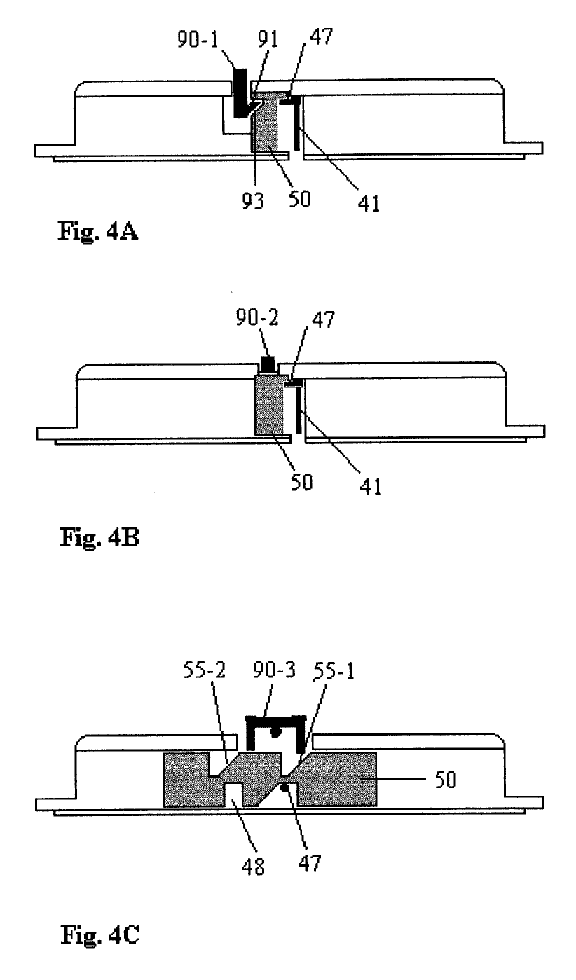

For example 1, the movement mechanism may be a spring mechanism as shown in FIG. 2. It is meant that the movement mechanism consists of a spring 19 and a locker 91. The spring 19 may form the punching status spring, which the spring is pushed or is pulled. The spring 19 disposed within the pod is power to move the action bar into the action groove 16 after the locker releases the spring from punching status. The locker 91 set between the action-bar and the pod locks the spring at punching status as shown in FIG. 4A. The trigger button 90-1 runs through the small open 21 of the top member 20. Here, the trigger button 90-1 has the locker 91, so movement of the trigger button 90-1 brings the locker 91 moving down. The locker 91 is in the pod and comes into the action groove to block the movement of the action bar at initial. The block is done by the locker 91 either through comes into a locker-site 93 on the surface of action-bar or through sits on the front of the action bar. When the trigger button 90-1 pushed down, the locker 91 lefts said locker-site 93 to release the action bar.

The release means the action bar 50, which powered by the spring 19, moves into the action groove 16 to perform the assembly.

For example 2, a rubber band may be another power means for the assembly. The rubber band at pulled status sets between the end of the action bar and the pod. Said end of the action bar and pod may have band-slots for engaging the rubber band (not shown). If the rubber band is located between the end of the action bar and the front site of the pod (at the front of the action bar), the action bar 50 moves forward. If the rubber band is located between the end of the action bar and the rear site of the pod, the action bar moves reverse toward the wall of the pod.

Alternatively, any suitable power means may be used to move the action-bar in the pod in accordance with the invention. The power means is not limited the spring or the rubber ring. The power means may include an electric power, an air power and a hydraulic power to move the action-bar. This kind of power means is performed the action-bar by pushing a start trigger button 90-2 (shown in FIG. 4B). The start trigger button may be not has the locker 91.

In one suitable approach, the assembly does not have the movement mechanism within the pod 30. The movement of action-bar is made by a push power from outside of the assembly. The push power to the action-bar includes user's hand or a movement mechanism of operating system located at out side of the assembly. For Example, the action bar 50 has a slid-handle on its top surface. The movement of action bar is followed the slid-handle when the slid-handle is moved toward the protruding of needle 47 via a push power from outside of the assembly on it. In here, the slid-handle fits in a small grip at the upper of the action groove 16 and runs through in the small grip (not shown).

General, performing the assembly has two options. One option is used user's hand manually that either pushes the trigger button 90 or moves the slid-handle. Second option is used by a perform-member 112 of an associated device. The associated device could perform the assembly by either pushes the trigger button or moves the slid-handle. When the assembly associates with the associated device, it is for convenient using the assembly. The assembly may be has a loading connector-site 32 on the pod 30. The loading connector-site 32 enables the assembly to be loaded to the associated device 110. Said perform-member 112 of associated device 110 performs the assembly 100. Further, the loading connector-site 32 has an electric bar 31 for transfer the electric signal to the associated device when the assembly stores a biosensor inside (detail shown late).

In the vertical case, the push-end of needle is directly pushed through the small open 21 in the top member.

In one suitable approach, power in the vertical direction is applied to the needle by a push-member. The push-member includes the trigger button and the perform-member from the associated device. The depth the needle inserts into the subject depends on the distance the push-member moves down. But the depth is no more than the length of the short-arm of needle 40. 1. When the push-end of the needle has the protrusion 47 on the long-arm, the needle is inserting by the protruding be pushed. 2. When the push-end of needle does not have the protrusion 47, the needle is inserted into the subject by pushed the needle.

In the inventive assembly having the springy extendable/retractable punching mechanism, there is no independent needle handle, no risk of accidental needle-stick, and no risk of biological contamination from the insertion site. The resilient extendable/retractable needle may be immediately concealed after performing the punching function of the assembly; the assembly has fewer traumas and the ability to protect the wound of the punch site and to stop bleeding from the wound of the punch site in a live subject. The construct of the inventive assembly is a simple mechanism, which reduces the number of malfunctions suffered by the assembly and enables the assembly to be a thin and lightweight article that provides is easy to adhere to the subject surface. The center portion of the assembly can be made from a firm material for housing the needle. The rest of the assembly can be made from a flexible material, which makes the assembly easy to apply to a subject surface (e.g., like a bandage). The assembly is covered with a protective package after being assembled. The assembly easily remains sterile after it is sterilized, for example sterilized by gamma irradiation.

The inventive assembly has an ability to perform the after the needle 40 is punched. In some cases, needle insertion into a subject is already considered a medical application (e.g., acupuncture). It means the assembly's preset application contained only the punch mechanism in it. Of cause, an application container configured for a test or a delivery task may be built in the pod. General, there are two kinds of preset application container that may be built into the assembly. One is for a delivery application that the application container storing substance to be delivered to the subject and the other is for a test application that application container storing test component to be reacted with sample from the subject. The pod 30 of the assembly uses the small groove for punching mechanism, so the pod has a lot of space to build the application container for purpose of test or delivery. The application container may be an open application container or a closed application container. It includes a syringe like mini-pump. Usually form of application container comprises the base member 10 and top member 20. Of course the invention also includes that the application container is an independent article laid into the pod.

In the case, the assembly in combination with the punch needle and the application container uses to perform the presetting application. The punch sections of assembly, including the extendable/retractable needle 40, the hole of base member 12, the ring member and the base member 10, are sections for connecting the application container.

Depending on the preset application in the assembly, both the pod 30 and needle 40 have variety features to fit the different purposes.

1. The resilient extendable/retractable needle includes a hollow hypodermic needle, a non-hollow needle, a blade needle, a biopsy needle and a test probe. Depending on the requirements of the preset application, the size of the needle is also variant size for different subject and for different purposes. According to the purpose of assembly, A. the springy extendable/retractable needle 40 is a hollow hypodermic needle that communication with application container (e.g. delivery or test container). The hollow hypodermic needle is used to insert into the subject for delivery substance or withdrawal sample. In further the hollow hypodermic needle includes hypodermic needle with two sharp ends 42. One sharp end inserts into the subject and other end for inserts into a closed application container to build communication with it. Whereas the communication means for said hollow hypodermic needle already communicated to application container or for said hollow hypodermic needle communicating to closed application container when said needle extension. B. The resilient extendable/retractable needle 40 is a non-hollow needle. It is used for punch the subject to perform assembly's task (e.g. pierced skin for testing or punched for acupuncture). C. The extendable/retractable needle is a blade needle with sharp edge that used for incision the subject or breaking package of subject to perform test function. D. The extendable/retractable needle is a biopsy needle with screw edge at the short-arm that used for taking tissue from the subject. E. The resilient extendable/retractable needle is a test probe, said test probe within absorption material inserting into subject to directly take sample from the subject.

2. The application container in the pod includes a delivery container and a test container. The application container, where housing filled injectable substance or arranged test components, connects to punching section of assembly. Said punching section includes the resilient extendable/retractable needle 40, the hole of base member 12, the ring member 39, and the subject surface of base member 10. When the application container is a penetrable closed container, the application container may be storing vacuum power or positive power too. Usually, the application container stable locates at the pod. But, when the application container set into the action bar, the action bar houses testing component or substance. This means the application container follows the action bar 50 to move in the pod.

Referring FIG. 5. In case of test application, the application container 70 for test task has a test chamber 71, and a test-duct 72. The inside of the test chamber 71 arranged test component 73. The test component 73 includes at least one of a strip, a chip, a test micro-array, and a biosensor. The test component may measure proteins, biological, chemical compositions, DNAs, and drugs. Most of strips, chips, test micro-arrays, and biosensors in current market are of thin and small size to be fit in the invention assembly.

The test-duct 72 builds a bridge between the subject and the test component 73. Means one end of test-duct opened at the punch section of assembly and other end of test-duct opened to test chamber. So the test-duct is path for transfer the sample from subject to the test chamber 71 and to test components 73.

About the test-duct 72, it is not only as a channel path for transfers the sample, but it also includes micro capillary and full absorption material in the test-duct for running sample to test component. For example: 1. The test-duct is micro capillary; the test chamber is open to outside by an air-tubing 76 for capillarity. 2. The test-duct is large duct within filled absorption material; the absorption material directly contacts the punch site of the subject.

According about the test-duct of the test container 70 built the path between the punch section and the test container. The test-duct 72 at the punch section of assembly, there may be a couple of options:

For example 1: the test-duct opened to the hole 12 of base member. The path of test-duct between the base member and the test chamber allows the test component of the test container 70 catches the sample from the punched site where is pierced by the needle 40.

For example 2: the test duct directly lied on the low surface of base member to touch the subject. The absorption member of the test-duct as the pad contacts the surface of subject to take sample directly from the surface of subject at pierced site.

For example 3: the test-duct connected to the hollow hypodermic needle or the test-duct is the hollow hypodermic needle. So the hollow needle is the path to drawn sample from the subject to the test component via a vacuum power. Said application container within test component may be a closed application container. The double ends needle is used to drawn sample (detail described in late).

For example 4: the test-duct connected to the test probe needle. So the probe directly takes the sample from the subject to the test component.

After the sample comes into the test chamber 71, and then reacts with regents of test component. The result will be shown to the user.

In one suitable approach, the application container may have a reading window 75 that being a transparent member at the top of the test chamber 71. The result is read by an optical instrument or by user's eye through the reading window 75 (e.g. shown color changed).

In anther suitable approach, the assembly is loaded to an associated device and transferred electric signal of test reaction to the associated device. It means the touched electric bars 31 send the electric signal. The touched electric bars 31 are at the connector-site 32 of the assembly and the couple-connector 111 of the associated device. The associated device via the electric bars touched connection would receive the electric signal, which is from the test component reacted with the sample.

In case of delivery application, the delivery container filled with substances 69 (usually, one-dose substance). The substance 69 is any injectable substance. The injectable means available injection material that includes liquid substance, encapsulation of substance, gel substance. The substances may be any kind medicines e.g. pharmaceutical drugs, protein drugs, DNA or RNA agents, vaccines, biological, biochemical agents and chemical agents. Usually the delivery container, which includes a syringe or a mini-pump, is communication to the hollow hypodermic needle. It is injected out said filled substance 69 into the subject.

In the invention assembly, the action-bar not only performs to pushing and concealing needle, but also may be perform more duty for the assembly.

In one suitable approach, the action bar 50 is a part of application container for performing preset application. The part means the action bar also being a plunger of pump and cooperated with another part of application container which being a barrel of pump.

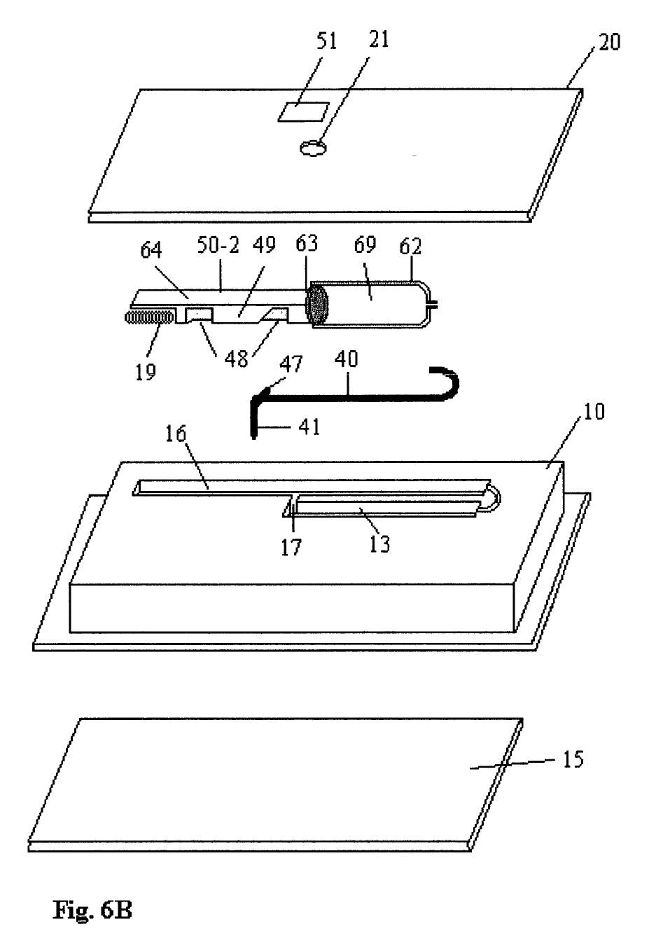

Here, the application container in the pod may be a mini-syringe or a mini-syringe like article a mini-pump 60. Referring FIG. 6A, 6B. The mini-pump 60 having a mini-barrel 62 with a mini-piston 63 and a mini-plunger 64 horizontally set into the pod 10. The barrel of mini-pump 62 has a first end and a second end. The first end of the barrel is communication to said punch section, usual communication to the resilient expendable/retractable hollow hypodermic needle 40 for the delivery or test task. The second end is open end in which the mini-piston 63 fit in. The mini-plunger 64 bringing the mini-piston 63 is moving into the mini-barrel. Because the mini-plunger is a part of the action-bar, the mini-plunger moves with the action-bar, called integral action-bar. It means movement of the integral action-bar does three things: inserting the needle, performing the mini-pump (e.g. delivering the substance or withdrawing sample), concealing the needle. An internal chamber 65 of mini-pump comprises the first end of mini-barrel 64 and the mini-piston 63. Followed said movement of integral action-bar, the movement of the mini-piston 63 in the mini-barrel 64 causes change of the internal chamber's pressure. The chamber's pressure is either position pressure or negative pressure. When the action-bar moves toward the needle end of mini-barrel, the chamber's pressure is position pressure. The position pressure allows to delivery the stored substance 69 from internal chamber of the mini-pump to the subject. When the action-bar moves toward the open end of mini-barrel, the internal chamber's pressure is negative pressure. The negative pressure allows drawing sample from subject to test components. Because the invention assembly is the wafer assembly, the cross section of barrel and the piston and the mini-plunger is not only circular shape but also is oval shape or rectangular shape at the cross section.

In one suitable embodiment of the integral action-bar, it is a U shaped integral action bar 50-1 that has two integral bars (shown in FIG. 6A). The first bar 56 of the integral action-bar uses to push the needle and the second bar 57, as the mini-plunger, uses to move the mini-piston of mini-pump. In the invention, the integral action bar 50-1 is not limited on the U shaped, including W, Y, shaped. So followed the movement of the integral action bar, the first bar of integral action-bar performs to insert the needle 40 and the second bar 57 moves into the mini-barrel of mini-pump that either pushes the mini-piston injection the substance into subject or pulls the mini-piston to withdraw the sample. When finished this, where the cavity 48 just at the protrusion of the needle, the needle automated retracts back from the subject to the pod.

In anther suitable embodiment, the integral action-bar is a single integral action-bar 50-2 that has both characters of the action-bar and the mini-plunger (shown in FIG. 6B). The integral bar 50-2 not only pushes the needle, but also performs the mini-pump. In here the second end of mini-barrel connected to the action-groove 16. The movement of single integral-bar 50-2 performs to insert needle into the subject, and then continue moves into the mini-barrel for application task.

The first end of the barrel may be a closed end to be communicated to the hollow hypodermic needle 40 when the needle extended. The closed mini-pump means that first end of barrel with a punch hole 68, which is sealed by a rubber member 67, and second end of barrel that is closed by the mini-piston. The double sharp ends hollow hypodermic needle fits the mini-pump. It is a hollow hypodermic needle with two short arms (shown in FIG. 3D). One of the two short arms is longer than other one. The punch hole 68 and the rubber member 67 are at the top of barrel to face the shorter short-arm. When the needle moves down, the longer short-arm inserts into the subject and the shorter short-arm runs thought the rubber member 67 into the punch hole 68 to be communicated to the mini-pump. It is means that the two hollow short-arms build the pathway between the subject and the closed mini-pump for delivery the filled substance or withdraw the sample.

Further, the first end of the barrel may be open to different punch sections of assembly. The punch sections include the low surface of base member 11, and the hole of base member 12. This kind mini-pump allows working at different place. Examples 1, it injects substance to the low surface of base member. The adhesion material (e.g. the dress portion 15) is available injected by the mini-pump. Examples 2, it produces a vacuum power at the needle punch site for taking sample (via either the test-duct or directly open the low surface of base member).

Further, the invention assembly builds more than one mini-pumps in the pod 30, called multi-pump. The integral action bar may be a W shape or a shark teeth shape. Examples 1, the chambers of mini-pumps are full different substances. When all of mini-barrels connect to the hollow hypodermic needle, the multi-pump with more than two different substances inject into the subject. Because each of these mini-pumps stored substance independently, the assembly 100 with the multi-pumps has advantage to delivery more than two substances, which could not be mixed in storage. Example 2, two mini-pumps of the assembly 100 stores two different substances that one mini-pump connects to the hollow hypodermic needle and one mini-pump opens to said low surface of base member.

According to mini-pump, usually the head of mini-plunger of integral action-bar, like convenient plunger, may be already set into the connection hole of the mini-piston, like the convenient piston. The mini-piston just follows the mini-plunger moving. For reduce pain of patient at punch, the integral action-bar moving in the pod to push the needle must be fast moved. The head mini-plunger and mini-piston, both are flat surface to be contacted. So the flat head of the mini-plunger could move fast at beginning to push the needle then push the flat min-piston.

In another suitable approach, the action-bar 50 services as whole application container for test or delivery task. This means the application container follows the action bar 50 to move in the pod.

1. In one embodiment of the integral action-bar, the integral action-bar 50-3 is a movable mini-pump lay and movement into the pod (shown in FIG. 7). It means that the integral action-bar 50-3 is the independent mini-pump article, which has the barrel, the piston, the plunger and the stored material (e.g. substance or test component). The integral action-bar 50-3 usually as a closed mini-pump as described before. Except the punch hole 68 and the rubber member 67 are at the front of barrel to face to the sharp end of long-arm. The double sharp ends hollow hypodermic needle may be a needle with double ends at the short-arm 41 and the long-arm 42. The double sharp ends hollow hypodermic needle maybe has a U turn at the end of long-arm 42 for fitting to arrange the closed mini-pump and the needle in the pod. The double sharp ends needle allows that one sharp end of short-arm inserts into said subject and other sharp end of long-arm inserts into the integral action-bar 50-3 through the rubber member 67. At beginning, the integral action-bar 50-3 is face to said horizontal sharp end of long-arm. There is a bit distance between the rubber member 67 and the shape end of long-arm. The assembly performs its function unless the integral action bar moving. It means that the integral action-bar 50-3 moves to push the needle insertion into subject and to be inserted into the chamber of mini-pump by the sharp end of long-arm. Then the barrel of the integral action-bar 50-3 stopped in the pod, let the piston could move in said mini-barrel.

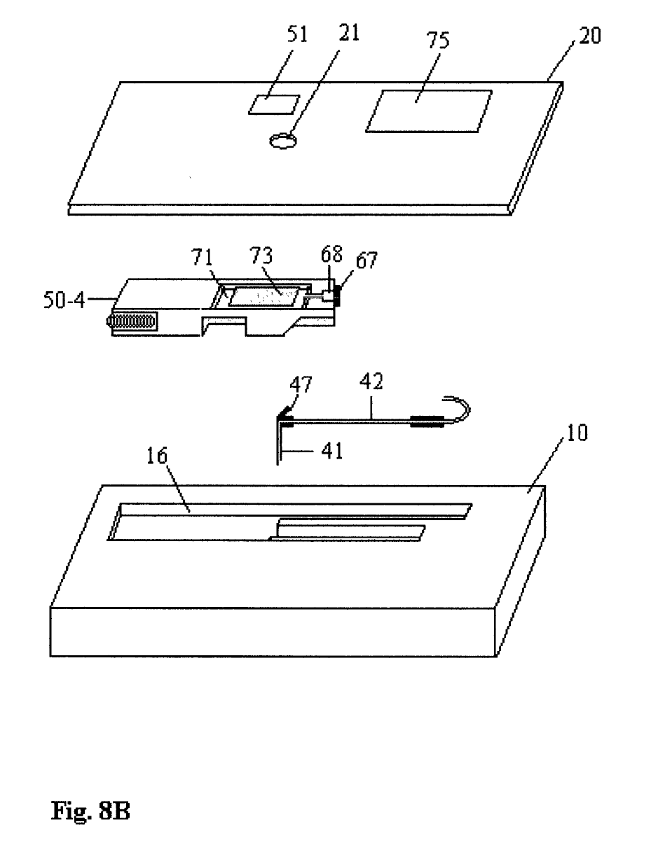

2. In embodiment of the integral action-bar 50-4, the integral action-bar is also a movable application container lay and movement into the pod (shown in FIGS. 8A,8B). The integral action-bar 50-4 usually is a closed application container for store materials. The punch hole 68 and the rubber member 67 are at the front of integral action-bar 50-4 to face to the sharp end of long-arm. So the double sharp ends needle allows that one sharp end of short-arm inserts into said subject and other sharp end of long-arm inserts into the closed integral action-bar 50-4 through the rubber member 67. The stored material in the closed application container not only is the substance 69 or test component 73 but also is the positive power or vacuum power.

For example 1, the integral action-bar as application container houses the testing component 73 and vacuum power. The stored vacuum power would take the sample from the subject to the testing component. For example 2, the integral action-bar as application container houses the substance and positive power. The stored positive power would inject the substance to the subject.

In further, the assembly is available used to be loaded an independent application container. In the case, the top member 10 of the pod has an opened space where uses for loading the independent application container. It means that the assembly may be available to use for a customized product to user. The independent application container includes both for delivery task (e.g. a filled substance barrel with flat piston) and for test task (e.g. a filled test-component container).

In further, the assembly may be combination with the test container and the mini-pump (one of embodiment as shown on FIG. 5B). 1. It is possible that the test container is an integrated part of mini-pump's barrel to take the sample from the hollow needle. 2. It is possible that the test container located between the hollow needle and mini-pump. 3. It is possible that the test container and mini-pump are directly open to same position of punch section e.g. the hole 12 of base member. However, the vacuum power of internal chamber directly is used to suck sample from said subject. The mini-barrel of vacuum pump maybe has a hole on its wall to open to outside of assembly. When the mini-piston moves to pass the hole, the internal chamber of the mini-pump opens to outside and stops suck.

In further, in some case the assembly having resilient extendable/retractable needle maybe perform with two steps for some application task. It is base on the special requirements for some kind application. For example, acupuncture is sent the needle into acupuncture point of the patients for therapy. The acupuncture needle needs stay in the subject long time until finish the therapy. Here, step 1 is actuating the extension let the acupuncture needle stays in the subject. Step 2 is actuating the retraction let the acupuncture needle conceals back into the pod. The acupuncture needle includes the blunt needle to press the surface of the acupuncture point.

When the assembly needs two steps for complete performance, a trigger button 90-3 has two push-members (push-member 1 and push-member 2). In one suitable approach, the trigger button 90-3 fixed to the top surface of the small open 21 by a joined protrude member. Depending on the joined protrude member, the push-member 1 and push-member 2 could be up-down move at the top surface of the assembly. Referring FIG. 4C, the assembly has the action-bar set in the pod. The action-bar has the basic configurations that described before, and addition configurations. The addition configurations just fit the trigger button 90-3. Said addition configurations mean that there are two cutouts with biased shaped surface (55-1, 55-2) at the top surface of the action-bar. Said the pus-member 1 pushed the cutout1 55-1. The action-bar moves in the pod to push the needle extension and exposed the cutout2 55-2 to the push-member 2. Said the push-member 2 pushed the cutout2 let the action-bar moves to conceal the needle in the pod.

Of cause, the trigger button 90-3 maybe directly pushes the needle by its push-members. Said push-member 1 moved down to directly pushes the needle, let the needle extends out the pod. Said push-member 2 pushed down, the needle retracts in the pod via the push-member 1 back.

General the process of perform inventive assembly comprising the steps of:

1. Applying the assembly to punch area of subject.

2. Performing the resilient extendable/retractable punching mechanism of assembly in one continuous motion, which includes performing function of the extendable/retractable needle and of the preset application in said assembly.

3. If the assembly having the dress portion 15, the assembly may be as a bandage retained on the punched site. Said assembly struck or fused on said subject to protect the punched site to avoid contamination.

Further, performing the resilient extendable/retractable punching mechanism of assembly also includes two steps for a preset application configuration. Said step-1 actuating said extension of needle to stay in the subject until said step-2 actuating said retraction of needle.

As description, the assembly not only allows dressing on the,subject, inserting the needle into the subject and concealing the needle to safe position after insertion done, but also maybe allows performing the presetting application and protecting the punch site. The assembly may avoid any risks associate with the needle-stick, the contamination and cross-infection in the operating process, it provides a safe and convenient article for healthcare.

The invention assembly involves a method that combines with an associated device or a complete automated device to perform the test or delivery task by using the assembly. The device is a tool for easy use the assembly. More important the assembly solves the problems of safety in performing process. The assembly may have the follow useful and convenient features.

First, the assembly has a readable marker 23 (e.g. barcode) that provides information about assembly. For example: when the assembly within the test application, the information includes an identification number of assembly, a presetting application class, detail name of components of application, requirement of sample for test component, wavelength of measuring test components; and when the assembly within the delivery application, the information includes an identification number of assembly, a presetting application class, detail name of drug, dose of drug, delivery place etc.

Second, the user easy uses the assembly association with a device that has measuring system and/or operating system. The device includes both a portable and a fixable device I. The assembly is measured by the measuring system of the device. For example, an optical instrument, which is one kind of the measuring system, reads the assembly. Said optical instrument has a light source, a detector (signal collector), filters and a computer. It catches the image data from the assembly through said reading window 75. Then the information analyzed by computer reports to users. The optical instrument has an interface that connects to Internet source and allows input of relative information and output of applicable information.

II. The assembly is operated by the operating system of the device. The assembly has the connector-site 32 for being loaded to the associated device (As shown in FIG. 1B, FIG. 3A). Said connector-site 32 may be shallow cavities on the outside of pod 30 for coupled to the associated device. The operating system of device 110 is an operating tool for the assembly. The operating system has at least one couple-connector 111 and a perform-member 112. Said couple-connector 111 uses to couple the connector-site of the assembly and to load the assembly on the device. Then said perform-member 112 moves to perform function of assembly. Movement of perform-member 112 makes active the assembly that either pushes said trigger button or directly pushes said needle or moves said action-bar. This means that said perform-member 112 may move at up down or horizontal. Final the couple-connector 111 maybe moves out at loaded position to disconnect the connector-site 32 and to release assembly. If the assembly with the dress portion 15, it retains on the subject to protect the punched site. Third, the assembly provides a method and apparatus for complete automated system or robot to perform delivery application and/or test application in the fields of medicine and biotechnological industry.

For example, the process of perform the assembly with complete automated system or robot comprising the steps:

a. automated loading a assembly to said complete automated system or robot; if said assembly has a barcode, information about requirement of presetting application is catch by reading said barcode;

b. automated applying said assembly to a punching area of subject where insertion site of needle fits the requirement of presetting application. If insertion site is a blood vessel or blood vessel free in human subject, the site may be determined by detection mechanism of complete automated system or robot;

c. automated performing function of said assembly which includes performing said resilient extendable/retractable needle and said preset application by operation mechanism of complete automated system or robot; If said assembly has preset test application inside the assembly, said complete automated system or robot would catch the test result.

d. automated disconnecting assembly from said operation mechanism of complete automated system or robot and releasing the assembly. The disconnecting assembly may be continuing dress on said subject.

Whereas the present invention wafer assembly allows dress on the punch site of subject to avoid potential risks of cross-infection and contamination associated with biological agents from subject e.g. blood and body fluid and allows insertion into the subject with resilient extendable/retractable needle mechanism to avoid potential risks associated with needle-stick and needle irritation.

What is claimed is:

1. A wafer assembly comprising:
a housing pod;
a resilient needle disposed within housing pod, said needle comprising at least one short-arm and at least one long-arm, and said needle being able to resiliently extend from said housing pod to puncture a subject and being able to resiliently retract within said housing pod after puncturing;
an application container configured for performing a healthcare task, said healthcare task including a test or delivery task; and
an actuation mechanism that actuates said extension of said needle and performance of said healthcare task.

2. The assembly of claim 1, wherein said actuation mechanism comprises an action-bar disposed within said housing pod, said action-bar having a push-part, said push-part being a protrusion for pushing said needle and a cavity for concealing said needle, wherein movement of said action-bar actuates said extension of said needle.

3. The assembly as defined in claim 1 wherein said needle is a hollow hypodermic needle in communication with said application container.

4. The assembly as defined in claim 1 wherein said needle is a non-hollow needle for puncturing said subject.

5. The assembly as defined in claim 1 wherein said needle is a needle having a sharp edge or point for piercing the surface of subject.

6. The assembly as defined in claim 1 wherein the needle is a test probe for directly take a sample.

7. The assembly as defined in claim 1, wherein comprising an adhesive dress portion on a bottom surface of said housing pod for applying said assembly to the subject surface.

8. The assembly of claim 1, further said needle being spring loaded to enable said resilient extension and retraction.

9. The assembly as defined in claim 1, further comprising a thin, penetrable membrane that seals said hole in said base member.

10. The assembly as defined in claim 1, further comprising a ring member in said housing pod for guiding said extension of said needle.

11. The assembly as defined in claim 1, further comprising a thin pad disposed around said hole for absorbing fluid.

12. The assembly of claim 2, wherein said movement of said action-bar is powered by at least one of a spring power, a rubber band power, an electric power, an air power, and a hydraulic power.

13. The assembly as defined in claim 2, wherein said action-bar is a part of said application container, said action-bar being integrated as a plunger of a pump within said application container, said plunger being cooperated with a barrel of pump within said application container.

14. The assembly as defined in claim 2, wherein said action bar serves as said application container for said test or delivery task.

15. The assembly as defined in claim 7, wherein said adhesive dress portion includes antibiotic and/or anesthetic reagents.

16. The assembly as defined in claim 1, wherein said application container comprises means for storing a test component, said test component being reacted with sample taken or withdrawn from said subject.

17. The assembly as defined in claim 1, wherein said application container comprises means for storing a substance to be delivered to said subject.

18. The assembly according to claim 1 further comprising a barcode on said assembly that provides information on said assembly that information includes at least one of a type of application container disposed within said assembly, a type of needle disposed within said assembly, a type of material to be delivered using said assembly, and a type of said test component stored within said assembly.

19. The assembly of claim 1, further comprising a loading connector-site on said housing pod that enables said assembly to be loaded to an associated device.

20. A method for using the assembly of claim 1:
applying said assembly to said subject;
actuating said extension of said needle and performance of said test or delivery task and retracting of said needle within said housing pod in one continuous motion.

21. A method for using the assembly within a resilient extendable/ retractable needle:
applying said assembly to said subject;
actuating said extension of said needle and maintaining said needle in an extended position within said subject or at said subject; and
enabling said needle to resiliently retract within said housing pod.
